(19) United States
(12) Patent Application Publication
(10) Pub. No.: US 2026/0315362 A1
(43) Pub. Date: Oct. 15, 2026

(54) PHARMACEUTICAL OR COSMETIC COMPOSITION FOR REDUCING SEBUM PRODUCTION ON THE SCALP

(71) Applicant: SOCIETE D'EXPLOITATION DE PRODUITS POUR LES INDUSTRIES CHIMIQUES SEPPIC, Paris Cedex 07 (FR)

(72) Inventors: Catherine Kern, Castres (FR); Stéphanie Dudonne, Castres (FR); Julien Fouilland, Pontrieux (FR)

(73) Assignee: SOCIETE D'EXPLOITATION DE PRODUITS POUR LES INDUSTRIES CHIMIQUES SEPPIC, Paris Cedex 07 (FR)

(21) Appl. No.: 19/481,527

(22) PCT Filed: Apr. 29, 2024
(86) PCT No.: PCT/EP2024/061764

§ 371 Date: Nov. 4, 2025

(30) Foreign Application Priority Data
May 5, 2023 (FR) 2304534

(51) Int. Cl. A61K 8/68; A61K 8/23; A61K 8/24; A61K 8/25; A61K 8/36; A61K 8/37; A61K 8/55; A61K 8/60; A61K 8/67; A61K 8/73; A61K 8/9789; A61Q 19/00

(52) U.S. Cl. CPC A61K 8/68; A61K 8/23; A61K 8/24; A61K 8/25; A61K 8/361; A61K 8/375; A61K 8/553; A61K 8/602; A61K 8/673; A61K 8/675; A61K 8/676; A61K 8/678; A61K 8/732; A61K 8/9789; A61Q 19/008; A61K 2800/92

(57) ABSTRACT

Composition (C) comprising, for 100% by mass: from 20% by mass to 80% by mass of at least one sphingolipid (a1), from 10% by mass to 50% by mass of digalactosyldiacylglycerol (a2), and from 1% by mass to 30% by mass of at least one phospholipid (a3), for use in a therapeutic treatment method for preventing or limiting sebum production on the scalp. Pharmaceutical composition (Cp) and food supplement (Ca) comprising said composition (C). Non-therapeutic use of said food supplement (Ca) and non-therapeutic method of implementation.

### TECHNICAL FIELD

The invention is situated within the fields of cosmetics and pharmaceuticals. It relates to the treatment of the scalp and more particularly to the production of sebum at the scalp.

### BACKGROUND

The scalp comprises all the tissues covering the cranium. It covers an area of about 600 cm² over a thickness of 6 millimeters. It differs from the skin on the rest of the human body in the presence of a very great number of pilo-sebaceous follicles—between 100 000 and 200 000 depending on the individual—from which the hair grows.

The hair fiber consists of a visible stem, a hair bulb located under the surface of the scalp, and a dermal papilla located at the base of the bulb. The stem consists of three concentric layers: the medulla, the cortex and the cuticle. The medulla is the central part, while the cortex is the middle layer and constitutes the body of the hair. The cortex is composed of long keratin chains that give the hair its elasticity, flexibility and strength. The hair fiber is protected by the cuticle, formed by scales of keratin sealed to one another by an intercellular cement which is rich in lipids and especially in ceramides. These lipid substances ensure the barrier function of the hair by giving it a certain impermeability.

At its base, the stem is surrounded by two sheaths: the outer epithelial sheath, which has a composition similar to the epidermis, and the inner epithelial sheath, which is rich in glycoproteins, constituting the nourishment base of the developing hair. The hair bulb is composed of keratinocytes, whose active division is responsible for the formation of the hair shaft, and of the inner and outer epithelial sheaths. The dermal papilla is a highly vascularized area and is home to the cell multiplication which is necessary for hair growth. It is composed of fibroblasts and ensures the nutrition, irrigation, oxygenation and removal of cellular waste from the hair.

The scalp is highly vascularized, because of the high density of hair follicles, and also includes a large number of nerve endings. Like the skin, the scalp is composed of three consecutive layers: the epidermis (the upper layer), the dermis and the hypodermis.

### DESCRIPTION

The epidermis, in constant renewal, consists of several layers of keratinocytes. These cells divide, differentiate and migrate from the deepest layer (basal layer) to the superficial layer (corneum or stratum corneum), in a process known as keratinization, then die and are eliminated. The corneum results from the superposition of 15 to 20 layers of completely keratinized dead cells: the corneocytes. These cells are integrated into an intercellular lipid structure called the “intercorneocyte cement”, composed of ceramides, fatty acids, triglycerides and cholesterol, ensuring a barrier role that maintains skin moisturization.

In the natural desquamation process, corneocytes detach from one or more layers of the stratum corneum, while a layer of cells in the basal layer divides to maintain the structure of the stratum corneum.

The sebaceous glands are present on almost the entire surface of the human body but in higher concentrations on the skin of the face (from 300 to 900/cm²) and on the scalp (about 200/cm²). Under normal conditions, the sebaceous glands produce and secrete sebum (0.7 gram to 1 gram per 24 hours), a complex fatty substance composed of fatty acids, triglycerides, wax esters, squalene and cholesterol.

Sebum participates in the formation of a hydrolipidic film covering the scalp, in association with the perspiration and the cellular debris from keratinization and desquamation. This film spreads at the base of the hair and forms a protective barrier against dehydration and infection by bacteria and fungi. The sebum thus lubricates the skin to protect it from friction and makes it more waterproof. It also promotes the survival of the commensal flora. In addition, the sebum transports fat-soluble antioxidants and retains them on the surface of the skin, thus giving rise to a protective activity against natural light. Lastly, it has an antibacterial and anti-inflammatory activity, and actively participates in the process of healing.

Excessive production of sebum is called hyperseborrhea. This condition is associated in humans with various factors such as an imbalance of thyroid and pituitary hormones, a metabolic disorder linked to the ingestion of saturated fats, intestinal or liver problems modifying the composition of the sebum, or else poor hygiene of the skin and scalp.

In addition, the proliferation of certain microorganisms (bacteria or fungi) can cause inflammation of the skin and scalp that promotes the production of sebum. In particular, the proliferation of the parasite Demodex folliculorum, a vermiform mite living in the hair follicles, can also trigger hyperseborrhea. Excessive sebum build-up on the scalp, combined with sweat, dead skin cells and other debris, can cause desquamation and itching and make the hair greasy. If left untreated, hyperseborrhea of the scalp can lead to dandruff or acne formation, to local skin infection (eczema, psoriasis, seborrheic dermatitis) and even to hair loss.

Scalp sebum content may be measured by means of a Sebumeter®. Technically, sebum is collected using a cassette equipped with a transparent film sensitive to sebaceous secretions. The measuring head of the cassette is inserted into the main unit of the instrument, where a photocell measures the transparency. The luminous transmission represents the sebum content at the surface of the measurement zone, expressed in μg of sebum/cm² of the scalp.

The American patent application published under number US 2009/286871 A1 discloses the cosmetic, pharmaceutical or dermatological use of a plant oil-based product as an agent for increasing lipid synthesis by the skin.

The American patent published under number U.S. Pat. No. 5,683,684 A discloses the use of a sphingolipid or a sphingolipid analogue as an active ingredient in the preparation of a cosmetic or dermopharmaceutical composition intended to protect the skin and/or hair against the harmful effects of pollution of the atmosphere with heavy metals.

The international application published under number WO 99/65459 A1 discloses cereal extracts comprising sphingolipids, glycoglycerolipids (including digalactosyldiglycerides or DGDG), triglycerides and phospholipids and their use for the manufacture of a dermatological, dermocosmetic or pharmaceutical composition, for purposes of anti-ageing treatment or for hydration of the skin or as an anti-inflammatory.

In the context of their research aimed at developing the use of natural compounds in therapy and cosmetics, the inventors have demonstrated that the use of a composition derived from wheat gluten extracts, administered orally, is able to regulate sebum production on the scalp, especially in people afflicted with hyperseborrhea.

Thus, according to a first aspect, a subject of the invention is a composition (C) characterized in that it comprises a wheat gluten extract comprising per 100% by mass:
from 20% by mass to 80% by mass of at least one sphingolipid (a1) resulting from the amidation of a fatty acid with sphingosine,
from 10% by mass to 50% by mass of digalactosyldiacylglycerol (a2), and
from 1% by mass to 30% by mass of at least one phospholipid (a3), and in that the sum of the mass contents of said at least one sphingolipid (a1) resulting from the amidation of a fatty acid with sphingosine, said digalactosyldiacylglycerol (a2) and said at least one phospholipid (a3) is equal to 100% by mass,
for use thereof orally, in a method of therapeutic treatment of the scalp to limit the production of sebum thereon.

“Sphingolipids” for the purposes of the present invention are lipid complexes resulting from the amidation of a fatty acid with sphingosine. Sphingosine is an amido-diol possessing an ethylenic double bond, called 2-aminooctadec-4-ene-1,3-diol and of formula (I):

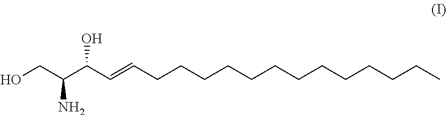 [SMILES: CCCCCCCCCCCCC/C=C/[C@@H](O)[C@@H](N)CO]

“Phospholipid” for the purposes of the present invention denotes a lipid comprising a phosphoric acid group in the monoester or diester form, and more particularly phosphoethanolamines, phosphatidylinositols, lysophosphatidylethanolamines, phosphatidic acids, phosphatidylcholines and lysophosphatidylcholines.

According to a first particular aspect, the composition (C) as defined above comprises per 100% by mass from 20% by mass to 60% by mass of at least one sphingolipid (a1).

According to a second particular aspect, the composition (C) as defined above comprises per 100% by mass from 19% by mass to 50% by mass of digalactosyldiacylglycerol (a2).

According to a third particular aspect, the composition (C) as defined above comprises per 100% by mass from 1% by mass to 10% by mass of at least one phospholipid (a3).

A subject of the invention more particularly is a composition (C) comprising per 100% by mass:
from 20% by mass to 60% by mass of at least one sphingolipid (a1) resulting from the amidation of a fatty acid with sphingosine,
from 19% by mass to 50% by mass of digalactosyldiacylglycerol (a2), and
from 1% by mass to 10% by mass of at least one phospholipid (a3),
for use thereof in a method of therapeutic treatment for preventing or limiting the production of sebum on the scalp.

According to another particular aspect of the invention, the composition (C) as defined above is in oily form or in pulverulent form.

Said composition (C) as defined above is prepared via methods known to those skilled in the art and more particularly the method described in the international patent application published under number WO 99/65459, on pages 8 to 11. It is prepared more particularly by a process comprising the following successive steps:
at least one step a) of provision of wheat gluten flour,
at least one step b) of addition of the wheat gluten flour to ethanol (97% minimum) with mechanical stirring for a time of at least two hours and at a temperature of 60° C.,
at least one step c) of draining followed by a sub-step of settling the mixture obtained in step b) so as to recover a homogeneous liquid phase,
at least one step d) of vacuum distillation of the liquid obtained in step c) at a temperature of 35° C. or less to remove the ethanol so as to obtain a concentrated composition,
at least one step e) of addition of the product obtained at the end of step d) to ethanol, with mechanical stirring and at a temperature of 4° C., so as to precipitate the medium and recover the liquid part,
at least one step f) of distillation of the liquid obtained at the end of step e) to remove the ethanol and obtain an oily composition,
at least one step g) of addition of the composition (C) to acetone so as to obtain a precipitate,
at least one step h) of filtration of the medium obtained at the end of step g) to recover a pulverulent solid,
drying of the solid cake obtained at the end of step g) to obtain a composition in powder form.

Another subject of the present invention is an edible pharmaceutical composition (Cp) comprising per 100% by mass:
from 10% by mass to 30% by mass of a composition (C) comprising per 100% by mass:
   from 20% by mass to 80% by mass, more particularly from 20% by mass to 60% by mass, of at least one sphingolipid (a1) resulting from the amidation of a fatty acid with sphingosine,
   from 10% by mass to 50% by mass, more particularly from 19% by mass to 50% by mass, of digalactosyldiacylglycerol (a2), and
   from 1% by mass to 30% by mass, more particularly from 1% by mass to 10% by mass, of at least one phospholipid (a3), the sum of the mass contents of said at least one sphingolipid (a1) resulting from the amidation of a fatty acid with sphingosine, said digalactosyldiacylglycerol (a2) and said at least one phospholipid (a3) being equal to 100% by mass and said composition (C) being a wheat gluten extract,
and
   from 70% by mass to 90% by mass of at least one pharmaceutically acceptable edible technological additive,
for use thereof orally, in a method of therapeutic treatment of the scalp to limit the production of sebum thereon.

According to one particular aspect, the therapeutic treatment method defined above is implemented on people afflicted with hyperseborrhea.

“Technological additive” denotes any chemical substance or any chemical composition whose technical function is to enable and/or to facilitate the mixing of the various constituents of said edible composition, to facilitate and/or to optimize the physical properties of said edible composition, such as to facilitate and/or to optimize its flow, its stability and its incorporation into a subsequent pharmaceutical and/or nutritional formulation, and which are able to meet the conditions required by the regulations in force for the marketing of a pharmaceutical formulation and/or a nutritional formulation.

The term “pharmaceutically acceptable technological additive” denotes a technological additive whose use meets the requirements of the pharmaceutical regulations in force in a country concerned.

According to one particular aspect of the invention, the edible technological additive is pharmaceutically acceptable and is selected from a diluent, a flow agent, a binder or a disintegrant.

A diluent is for example lactose, sucrose, saccharose, glucose, maltodextrin, mannitol, sorbitol, xylitol, isomalt, calcium hydrogenphosphate, microcrystalline cellulose, starches such as corn starch, wheat starch or potato starch, dicalcium phosphate, sodium carbonate, calcium carbonate or magnesium carbonate, or monoglycerides and/or diglycerides of fatty acids comprising from 8 to 24 carbon atoms.

A flow agent is for example magnesium stearate, talc, sodium stearylfumarate, hydrogenated vegetable oils, anhydrous colloidal silica, sodium benzoate or silica dioxide.

A binder is for example starches in the form of starch paste, pregelatinized starches, hydroxypropylmethyl cellulose (HPMC), methyl cellulose, sucrose syrups or acacia gum.

A disintegrant is for example starches, sodium starch glycolate, alginic acid, sodium alginate, sodium croscarmellose, crospovidone and/or polyvinylpyrrolidone.

According to one particular aspect of the invention, said edible pharmaceutical composition (Cp) as defined above further comprises at least one active principle chosen from bioactive lipids, water-soluble or water-dispersible trace element salts, water-soluble or fat-soluble vitamins, prebiotics, probiotics, milk proteins and/or milk protein concentrates, plant or animal enzymes, amino acids, peptides, sugars, flavor enhancers and flavoring agents.

Bioactive lipids optionally present in said edible pharmaceutical composition (Cp) as defined above are for example phytosterols, such as those extracted from vegetable oils, and more particularly extracts of sea buckthorn oil, of corn oil or of soybean oil; complexes of phytosterols isolated from vegetable oils, such as cholestatin composed of campesterol, of stigmasterol and of brassicasterol; phytostanols; carotenoids, which belong to the family of the terpenoids, extracted from algae, green plants, fungi or bacteria; polyunsaturated fatty acids of the omega-3 group, such as alpha-linoleic acid, eicosapentaenoic acid or docosahexanoic acid; polyunsaturated fatty acids of the omega-6 group, such as linoleic acid, γ-linoleic acid, eicosadienoic acid, dihomo-γ-linoleic acid, arachidonic acid, docosadienoic acid, docosatetraenoic acid or docosapentaenoic acid.

Water-soluble or water-dispersible trace element salts optionally present in said edible pharmaceutical composition (Cp) as defined above are for example ferrous carbonate, ferrous chloride tetrahydrate, ferric chloride hexahydrate, ferrous citrate hexahydrate, ferrous fumarate, ferrous lactate tetrahydrate, ferrous sulfate monohydrate, ferrous sulfate heptahydrate, ferrous chelate of amino acid hydrates, iron glycine chelate; calcium iodate hexahydrate, anhydrous calcium iodate; sodium iodide, potassium iodide; cobalt acetate tetrahydrate, basic cobalt carbonate monohydrate, cobalt carbonate hexahydrate, cobalt sulfate heptahydrate, cobalt sulfate monohydrate, cobalt nitrate hexahydrate; cupric acetate monohydrate, basic copper carbonate monohydrate, cupric chloride dihydrate, copper methionate, cupric sulfate pentahydrate, cuprous chelate of amino acid hydrates, cuprous chelate of glycine hydrate, copper chelate of hydroxy analogue of methionine; manganous carbonate, manganous chloride tetrahydrate, manganese hydrogenphosphate trihydrate, manganous sulfate tetrahydrate, manganous sulfate monohydrate, manganese chelate of amino acid hydrate, manganese chelate of glycine hydrate, manganese chelate of hydroxy analogue of methionine; ammonium molybdate, sodium molybdate, sodium selenite, sodium selenate; the organic form of selenium produced by Saccharomyces cerevisiae; selenomethionine (inactivated selenium yeast); and the selenomethionine produced by Saccharomyces cerevisiae (inactivated selenium yeast).

Water-soluble or fat-soluble vitamins optionally present in said edible pharmaceutical composition (Cp) as defined above are for example vitamin A, more particularly in its form of retinol, retinyl acetate, retinyl palmitate or n-carotene; vitamin D2, more particularly in its form of ergocalciferol or 25-hydroxycalciferol; vitamin D3, more particularly in its form of cholecalciferol; vitamin K, more particularly in its form of phylloquinone (phytomenadione) or menaquinone; vitamin B1, more particularly in its form of thiamine hydrochloride, thiamine mononitrate, thiamine chloride monophosphate or thiamine chloride pyrophosphate, vitamin B2, more particularly in its form of riboflavin, riboflavin sodium 5′-phosphate; vitamin B6, more particularly in its form of pyridoxine hydrochloride, pyridoxine 5′-phosphate or pyridoxal 5′-phosphate; vitamin B12, more particularly in the form of cyanocobalamin, hydroxocobalamin, 5′-deoxyadenosylcobalamin or methylcobalamin; vitamin C, more particularly in its form of L-ascorbic acid, sodium L-ascorbate, calcium L-ascorbate, potassium L-ascorbate, calcium salts of palmityl-6-L-ascorbic acid, sodium ascorbylmonophosphate; pantothenic acid, more particularly in its form of calcium D-pantothenate, sodium D-pantothenate, dexpanthenol or pantethine; vitamin PP, more particularly in its form of nicotinic acid, niacin, nicotinamide or inositol hexanicotinate (inositol hexaniacinate); vitamin B9, more particularly in its form of folic acid, folates, more particularly in their form of pteroylmonoglutamic acid, calcium L-methylfolate, (6S)-5-methyltetrahydrofolic acid in the form of glucosamine salt, vitamin H2, B7 or BW, more particularly in its form of biotin; choline, more particularly in its form of choline chloride, choline dihydrogen citrate, choline bitartrate; inositol; carnitine, more particularly in its form of L-carnitine, L-carnitine-L-tartrate; taurine.

Prebiotics optionally present in said edible pharmaceutical composition (Cp) as defined above are for example inulin, transgalactooligosaccharides, fructans and mannooligosaccharides.

Probiotics optionally present in said edible pharmaceutical composition (Cp) as defined above are various strains of Saccharomyces cerevisiae, of Bacillus cereus var. toyoi, of Bacillus subtilis alone or in combination with Bacillus licheniformis, or else strains of Enteroccocus faecium. These strains of microorganisms are generally combined with a solid support, for example calcium carbonate, dextrose or sorbitol.

Proteins and/or protein concentrates optionally present in said edible pharmaceutical composition (Cp) as defined above are for example milk proteins obtained from milk, such as colostrum in the form of a lyophilized or atomized powder, whey in the form of a powder, and fractions which are purified or enriched in IgG, in lactoferrin or in lactoperoxidase.

Plant or animal enzymes optionally present in the edible pharmaceutical composition (Cp) as defined above are for example promutase, superoxide dismutase (SOD), 3-phytase, 6-phytase, endo-1,4-beta-glucanases, endo-1,4-beta-xylanases, or else other enzymes which improve or promote digestion.

Peptides optionally present in said edible pharmaceutical composition (Cp) as defined above are for example avocado peptides, lupin peptides, quinoa peptides, maca peptides, fermented or unfermented soybean peptides, rice peptides, peptides present in Acacia macrostachya seed extract or peptides present in passionflower seed extracts.

Amino acids optionally present in said edible pharmaceutical composition (Cp) as defined above are for example alanine, arginine, asparagine, aspartic acid, cysteine, glutamic acid, glutamine, glycine, histidine, isoleucine, leucine, lysine, methionine, phenylalanine, proline, hydroxyproline, pyrrolysine, selenocysteine, serine, threonine, tryptophan, tyrosine, valine, sarcosine or ornithine.

Sugars optionally present in said edible pharmaceutical composition (Cp) as defined above are for example water-soluble polysaccharides or sugars of low molecular weight, such as oligosaccharides or mono- or disaccharides, such as, for example, glucose, lactose or dextrose.

Flavor enhancers optionally present in the edible pharmaceutical composition (Cp) as defined above are for example glutamates, such as glutamic acid, monosodium glutamate, monopotassium glutamate, calcium diglutamate, ammonium glutamate or magnesium diglutamate; guanylates, such as, for example, guanylic acid (guanosine monophosphate), disodium guanylate, dipotassium guanylate or calcium guanylate, inosinates, such as, for example, inosinic acid, disodium inosinate, dipotassium inosinate or calcium inosinate, or else intense sweeteners such as Stevia extracts or rebaudiosides.

Said edible pharmaceutical composition (Cp) may be in solid or liquid form.

According to one particular aspect of the invention, said edible pharmaceutical composition (Cp) takes the form of a solid, and more particularly the form of a tablet, a hard capsule, a soft capsule, a powder, a film-coated tablet, a confectionery gum or a granule.

When the edible pharmaceutical composition (Cp) as defined above takes the form of a tablet, it preferably comprises from 50 mg to 150 mg of said composition (C) as defined above, per 500 mg tablet; when it takes the form of a soft capsule, it preferably comprises from 120 mg to 360 mg of said composition (C) per 1200 mg soft capsule; when it takes the form of a hard capsule, it preferably comprises from 10 mg to 30 mg of composition (C) per 100 mg capsule; when it takes the form of a powder, it preferably comprises from 200 mg to 600 mg of composition (C) per 2000 mg of powder.

When the edible pharmaceutical composition (Cp) takes the form of a powder, it is obtained by introducing its various constituents into a mixer equipped with at least one mechanical stirring system, such as for example flat or impeller-type stirring blades, and the mixer is optionally a tumbler mixer, and the mixer is optionally equipped with a lump breaker system. This mixing operation is generally carried out at ambient temperature, especially between 15° C. and 30° C.

When the edible pharmaceutical composition (Cp) takes the form of a confectionery gum, it preferably comprises from 150 mg to 450 mg of composition (C) per 1.5 g confectionery gum.

When the edible pharmaceutical composition (Cp) takes the form of a liquid, it preferably comprises from 100 mg to 300 mg of composition (C) per 1 cm³ ampoule.

Another subject of the invention is the non-therapeutic use by oral administration of an effective amount of a composition (C) comprising per 100% by mass:
from 20% by mass to 80% by mass, more particularly from 20% by mass to 60% by mass, of at least one sphingolipid (a1) resulting from the amidation of a fatty acid with sphingosine,
from 10% by mass to 50% by mass, more particularly from 19% by mass to 50% by mass, of digalactosyldiacylglycerol (a2), and
from 1% by mass to 30% by mass, more particularly from 1% by mass to 10% by mass, of at least one phospholipid (a3), the sum of the mass contents of said at least one sphingolipid (a1) resulting from the amidation of a fatty acid with sphingosine, said digalactosyldiacylglycerol (a2) and said at least one phospholipid (a3) being equal to 100% by mass and said composition (C) being a wheat gluten extract, for preventing or slowing the appearance of unsightly signs linked to the presence of excess sebum on the scalp,

A further subject of the invention is a method of cosmetic treatment for preventing or slowing the appearance of unsightly signs linked to the presence of excess sebum on the scalp, characterized in that an effective amount is administered orally of a composition (C) comprising per 100% by mass:
from 20% by mass to 80% by mass, more particularly from 20% by mass to 60% by mass, of at least one sphingolipid (a1) resulting from the amidation of a fatty acid with sphingosine,
from 10% by mass to 50% by mass, more particularly from 19% by mass to 50% by mass, of digalactosyldiacylglycerol (a2), and
from 1% by mass to 30% by mass, more particularly from 1% by mass to 10% by mass, of at least one phospholipid (a3), the sum of the mass contents of said at least one sphingolipid (a1) resulting from the amidation of a fatty acid with sphingosine, said digalactosyldiacylglycerol (a2) and said at least one phospholipid (a3) being equal to 100% by mass and said composition (C) being a wheat gluten extract.

Within the meaning of the present invention, the term “unsightly signs linked to the presence of sebum on the scalp” is understood to mean any modifications to the external appearance of the scalp that are due to an excess amount of sebum on said scalp, such as, for example, a glistening appearance, a greasy appearance, a sticky sensation of the scalp, or the presence of films. In addition, the sebum present in excess on the scalp can spread over the root of the hair and cause a greasy appearance to said hair.

Within the meaning of the present invention, the term “effective amount” denotes an amount which enables a consumer of average attention to notice visually the reality of the aesthetic appearance asserted, namely the absence of or reduction in a glistening appearance, and/or a greasy appearance, and/or a sticky sensation and/or the presence of films on the scalp.

The experimental study presented in the continuation of the present description reveals that an amount of between 1 mg and 250 mg, more particularly between 10 mg and 150 mg, more particularly still between 15 mg and 100 mg, and more particularly still between 30 mg and 70 mg of said composition, ingested daily, may be considered as an effective amount.

Another subject of the present invention is a food supplement (Ca) comprising per 100% by mass:
more than 0% by mass to 30% by mass of said composition (C) as defined above and
from 70% by mass to less than 100% by mass of at least one nutritionally acceptable technological additive.

Preferably, said food supplement (Ca) comprises per 100% by mass:
more than 0% by mass and less than 10% by mass of said composition (C) as defined above and
more than 90% by mass and less than 100% by mass of at least one nutritionally acceptable technological additive.

The term “food supplement” is understood to mean an edible composition whose purpose is to supplement the normal diet and which constitutes a concentrated source of nutrients or other substances having a nutritional or physiological effect alone or in combination. The food supplement makes it possible to prevent certain deficiencies or to respond to specific needs in the diet of an individual, in particular during physical exertion. This definition of “food supplement” is given in Article 2 of Decree No. 2006-352 of 26 Mar. 2006 of the French Republic concerning food supplements and in Directive 2002/46/EC of the European Parliament and of the Council of 10 Jun. 2002.

The term “nutritionally acceptable technological additive” denotes a technological additive as defined above which when used meets the requirements of the regulations relating to food supplements in force in a country under consideration.

According to one particular aspect of the invention, said nutritionally acceptable technological additive is chosen from a diluent, a flow agent, a binder or a disintegrant.

A diluent is for example lactose, sucrose, saccharose, glucose, maltodextrin, mannitol, sorbitol, xylitol, isomalt, calcium hydrogenphosphate, microcrystalline cellulose, starches such as corn starch, wheat starch or potato starch, dicalcium phosphate, sodium carbonate, calcium carbonate or magnesium carbonate, or monoglycerides and/or diglycerides of fatty acids comprising from 8 to 24 carbon atoms.

A flow agent is for example magnesium stearate, talc, sodium stearylfumarate, hydrogenated vegetable oils, anhydrous colloidal silica, sodium benzoate or silica dioxide.

A binder is for example starches in the form of starch paste, pregelatinized starches, hydroxypropylmethyl cellulose (HPMC), methyl cellulose, sucrose syrups or acacia gum.

A disintegrant is for example starches, sodium starch glycolate, alginic acid, sodium alginate, sodium croscarmellose, crospovidone and/or polyvinylpyrrolidone.

According to one particular aspect of the invention, said food supplement (Ca) as defined above further comprises at least one active principle chosen from bioactive lipids, water-soluble or water-dispersible trace element salts, water-soluble or fat-soluble vitamins, prebiotics, probiotics, milk proteins and/or milk protein concentrates, plant or animal enzymes, amino acids, peptides, sugars, flavor enhancers and flavoring agents.

Bioactive lipids optionally present in said food supplement (Ca) as defined above are for example phytosterols, such as those extracted from vegetable oils, and more particularly extracts of sea buckthorn oil, of corn oil or of soybean oil; complexes of phytosterols isolated from vegetable oils, such as cholestatin composed of campesterol, of stigmasterol and of brassicasterol; phytostanols; carotenoids, which belong to the family of the terpenoids, extracted from algae, green plants, fungi or bacteria; polyunsaturated fatty acids of the omega-3 group, such as alpha-linoleic acid, eicosapentaenoic acid or docosahexanoic acid; polyunsaturated fatty acids of the omega-6 group, such as linoleic acid, γ-linoleic acid, eicosadienoic acid, dihomo-γ-linoleic acid, arachidonic acid, docosadienoic acid, docosatetraenoic acid or docosapentaenoic acid.

Water-soluble or water-dispersible trace element salts optionally present in said food supplement (Ca) as defined above are for example ferrous carbonate, ferrous chloride tetrahydrate, ferric chloride hexahydrate, ferrous citrate hexahydrate, ferrous fumarate, ferrous lactate tetrahydrate, ferrous sulfate monohydrate, ferrous sulfate heptahydrate, ferrous chelate of amino acid hydrates, iron glycine chelate; calcium iodate hexahydrate, anhydrous calcium iodate; sodium iodide, potassium iodide; cobalt acetate tetrahydrate, basic cobalt carbonate monohydrate, cobalt carbonate hexahydrate, cobalt sulfate heptahydrate, cobalt sulfate monohydrate, cobalt nitrate hexahydrate; cupric acetate monohydrate, basic copper carbonate monohydrate, cupric chloride dihydrate, copper methionate, cupric sulfate pentahydrate, cuprous chelate of amino acid hydrates, cuprous chelate of glycine hydrate, copper chelate of hydroxy analogue of methionine; manganous carbonate, manganous chloride tetrahydrate, manganese hydrogenphosphate trihydrate, manganous sulfate tetrahydrate, manganous sulfate monohydrate, manganese chelate of amino acid hydrate, manganese chelate of glycine hydrate, manganese chelate of hydroxy analogue of methionine; ammonium molybdate, sodium molybdate, sodium selenite, sodium selenate; the organic form of selenium produced by Saccharomyces cerevisiae; selenomethionine (inactivated selenium yeast); and the selenomethionine produced by Saccharomyces cerevisiae (inactivated selenium yeast).

Water-soluble or fat-soluble vitamins optionally present in said food supplement (Ca) as defined above are for example vitamin A, more particularly in its form of retinol, retinyl acetate, retinyl palmitate or beta-carotene; vitamin D2, more particularly in its form of ergocalciferol or 25-hydroxycalciferol; vitamin D3, more particularly in its form of cholecalciferol; vitamin K, more particularly in its form of phylloquinone (phytomenadione) or menaquinone; vitamin B1, more particularly in its form of thiamine hydrochloride, thiamine mononitrate, thiamine chloride monophosphate or thiamine chloride pyrophosphate, vitamin B2, more particularly in its form of riboflavin, riboflavin sodium 5′-phosphate; vitamin B6, more particularly in its form of pyridoxine hydrochloride, pyridoxine 5′-phosphate or pyridoxal 5′-phosphate; vitamin B12, more particularly in the form of cyanocobalamin, hydroxocobalamin, 5′-deoxyadenosylcobalamin or methylcobalamin; vitamin C, more particularly in its form of L-ascorbic acid, sodium L-ascorbate, calcium L-ascorbate, potassium L-ascorbate, calcium salts of palmityl-6-L-ascorbic acid, sodium ascorbylmonophosphate; pantothenic acid, more particularly in its form of calcium D-pantothenate, sodium D-pantothenate, dexpanthenol or pantethine; vitamin PP, more particularly in its form of nicotinic acid, niacin, nicotinamide or inositol hexanicotinate (inositol hexaniacinate); vitamin B9, more particularly in its form of folic acid, folates, more particularly in their form of pteroylmonoglutamic acid, calcium L-methylfolate, (6S)-5-methyltetrahydrofolic acid in the form of glucosamine salt, vitamin H2, B7 or BW, more particularly in its form of biotin; choline, more particularly in its form of choline chloride, choline dihydrogen citrate, choline bitartrate; inositol; carnitine, more particularly in its form of L-carnitine, L-carnitine-L-tartrate; taurine.

Prebiotics optionally present in said food supplement (Ca) as defined above are for example inulin, transgalactooligosaccharides, fructans and mannooligosaccharides.

Probiotics optionally present in said food supplement (Ca) as defined above are various strains of Saccharomyces cerevisiae, of Bacillus cereus var. toyoi, of Bacillus subtilis alone or in combination with Bacillus licheniformis, or else strains of Enterococcus faecium. These strains of microorganisms are generally combined with a solid support, for example calcium carbonate, dextrose or sorbitol.

Proteins and/or protein concentrates optionally present in said food supplement (Ca) as defined above are for example milk proteins obtained from milk, such as colostrum in the form of a lyophilized or atomized powder, whey in the form of a powder, and fractions which are purified or enriched in IgG, in lactoferrin or in lactoperoxidase.

Plant or animal enzymes optionally present in said food supplement (Ca) as defined above are for example promutase, superoxide dismutase (SOD), 3-phytase, 6-phytase, endo-1,4-beta-glucanases, endo-1,4-beta-xylanases, or else other enzymes which improve or promote digestion.

Peptides optionally present in said food supplement (Ca) as defined above are for example avocado peptides, lupin peptides, quinoa peptides, maca peptides, fermented or unfermented soybean peptides, rice peptides, peptides present in Acacia macrostachya seed extract or peptides present in passionflower seed extracts.

Amino acids optionally present in said food supplement (Ca) as defined above are for example alanine, arginine, asparagine, aspartic acid, cysteine, glutamic acid, glutamine, glycine, histidine, isoleucine, leucine, lysine, methionine, phenylalanine, proline, hydroxyproline, pyrrolysine, selenocysteine, serine, threonine, tryptophan, tyrosine, valine, sarcosine or ornithine.

Sugars optionally present in said food supplement (Ca) as defined above are for example water-soluble polysaccharides or sugars of low molecular weight, such as oligosaccharides or mono- or disaccharides, such as, for example, glucose, lactose or dextrose.

Flavor enhancers optionally present in said food supplement (Ca) as defined above are for example glutamates, such as glutamic acid, monosodium glutamate, monopotassium glutamate, calcium diglutamate, ammonium glutamate or magnesium diglutamate; guanylates, such as, for example, guanylic acid (guanosine monophosphate), disodium guanylate, dipotassium guanylate or calcium guanylate, inosinates, such as, for example, inosinic acid, disodium inosinate, dipotassium inosinate or calcium inosinate, or else intense sweeteners such as Stevia extracts or rebaudiosides.

Said food supplement (Ca) may be in solid or liquid form.

Said food supplement (Ca) may be in any form of food product known to a person skilled in the art, such as a drink, and more particularly an aqueous drink, a solution, a fruit juice, a flavored drink, an energy drink, an alcoholic drink, a coffee-based drink, a chocolate-based drink, a tea-based drink, a milk product, and more particularly milk, yoghurt, a milk desert, drinkable yoghurt, a cheese, an ice cream, a chocolate bar, a cereal product, and more particularly a cereal bar, a biscuit, breakfast cereals, flours, bread-making products, a specialized nutrition product, more particularly an infant nutrition product, a nutrition product for preparing for physical exertion, a clinical nutrition product, a meal substitute, confectionery, more particularly chewing gums, bonbons, caramels, dragees, humbugs, marshmallows, lokums, nougats, fruit jellies, licorice.

The food supplement (Ca) according to the invention may also be in any presentation form known to a person skilled in the art, such as for example in the form of a tablet, a hard capsule, a soft capsule, a syrup, a powder, such as for example an immediate-release powder, a delayed-release powder or a powder for reconstituted drinks, a liquid, a stick, or a gel.

According to one particular aspect of the invention, said food supplement (Ca) as defined above takes the form of a solid, and more particularly the form of a tablet, a hard capsule, a soft capsule, a powder, a film-coated tablet, a confectionery gum or a granule.

When said food supplement (Ca) as defined above takes the form of a tablet, it preferably comprises less than 50 mg of said composition (C) as defined above, per 500 mg tablet; when it takes the form of a soft capsule, it preferably comprises less than 120 mg of said composition (C) per 1200 mg soft capsule; when it takes the form of a hard capsule, it preferably comprises less than 10 mg of said composition (C) as defined above per 100 mg capsule; when it takes the form of a powder, it preferably comprises less than 200 mg of said composition (C) as defined above per 2000 mg of powder.

When said food supplement (Ca) takes the form of a powder, it is obtained by introducing its various constituents into a mixer equipped with at least one mechanical stirring system, such as for example flat or impeller-type stirring blades, and the mixer is optionally a tumbler mixer, and the mixer is optionally equipped with a lump breaker system. This mixing operation is generally carried out at ambient temperature, especially between 15° C. and 30° C.

When said food supplement (Ca) is in the form of a confectionery gum, it preferably comprises less than 150 mg of said composition (C) as defined above per 1.5 g confectionery gum.

When the food supplement (Ca) is in the form of a liquid, it preferably comprises less than 100 mg of said composition (C) as defined above per 1 cm³ ampoule.

A further subject of the present invention is the use by oral administration of said food supplement (Ca) as defined above for preventing and/or slowing the appearance of unsightly signs linked to the presence of excess sebum on the scalp.

Another subject of the present invention is a method of cosmetic treatment for preventing or slowing the appearance of unsightly signs linked to the presence of excess sebum on the scalp, characterized in that an effective amount is administered orally of a food supplement (Ca) as defined above.

According to one particular aspect of the invention, said food supplement (Ca) as defined above is administered orally in a proportion of at least 15 mg, more particularly of at least 30 mg, of said composition (C) per day for at least 4 weeks, more particularly for at least 8 weeks, even more particularly for 12 weeks.

### EXAMPLES

The following examples illustrate the invention, though without limiting it.

### Comparative Formulations

A commercial wheat gluten extract is used in the form of a pulverulent composition (C) which comprises per 100% of its mass: 52.5% by mass of sphingolipids, 41.5% by mass of digalactosyldiacylglycerol and 6% by mass of phospholipids.

Two formulations were prepared, respectively a food supplement (Ca) (comprising the composition (C)) and a food supplement (C′a) (not comprising the composition (C)), which comprise the ingredients as indicated in Table 1:

TABLE 1

| | Amount per capsule | | |
|---|---|---|---|
| | | Food supplement | Food supplement |
| Components | | (Ca) | (C′a) |

| Composition | 15 | mg | — | |
|---|---|---|---|---|
| (C) | | | | |
| Maltodextrin | 230 | mg | 245 | mg |
| Dicalcium | 100 | mg | 100 | mg |
| phosphate | | | | |
| Magnesium | 50 | mg | 50 | mg |
| carbonate | | | | |
| Silica | 30 | mg | 30 | mg |
| Magnesium | 10 | mg | 10 | mg |
| stearate | | | | |
| Total net | 435 | mg | 435 | mg |
| weight | | | | |
| Total weight | 549 | mg | 549 | mg |
| (with capsule | | | | |
| weight) | | | | |

### Principle of the Evaluation Method

The dermo-purifying activity was evaluated by measuring the sebum content on the scalp of volunteers with a baseline level greater than 140 μg/cm², by sebumetry®, and also through a self-assessment form completed by the study subjects.

The sebum content of the scalp is measured by means of a Sebumeter®.

Technically, sebum is collected using a cassette equipped with a transparent film sensitive to sebaceous secretions. The measuring head of the cassette is inserted into the main unit of the instrument, where a photocell measures the transparency. The luminous transmission represents the sebum content at the surface of the measurement zone, expressed in μg of sebum/cm² of the scalp.

### Procedure

The technical effect of the composition (C) was demonstrated in a pilot, randomized, double-blind, parallel-group clinical study. This made it possible to test the effect of the daily consumption of 30 mg of (C) versus placebo over a period of 84 days (12 weeks).

Ethnically Caucasian women aged between 18 and 65 years, with telogen effluvium (proportion of telogen-phase hair >15%) and complaints of thin, brittle hair, and with a sebum level greater than or equal to 140 μg/cm², were included in the study. For 84 days (from DO to D84) they consumed either 2 capsules per day of the food supplement (Ca) or 2 capsules per day of the food supplement (C′a). The measurements were carried out on day 0 (DO) and at 56 (D56) and 84 days (D84).

### Results

The results obtained are set out in the table below.

TABLE 2

| | Food supplement (C′a) | | Food supplement (Ca) | |
|---|---|---|---|---|
| | (number of subjects = 11) | | (number of subjects = 14) | |
| | Mean +/− SDM | % VS | Mean +/− SDM | % VS D 0 |
| | (p vs D 0) | D 0 | (p vs D 0) | (p vs placebo) |
| D 0 | 166.4 ± 7.5 | — | 196.0 ± 12.1 | — |
| D 56 | 168.0 ± 7.0 | +2.3% | 185.1 ± 9.9 | −4.2% |
| D 84 | 168.5 ± 7.4 | +2.8% | 171.7 ± 11.4 | −11.9% |
| | | | (p = 0.013) | (p = 0.044) |

SDM: standard deviation from the mean

### Observations and Comments

A significant decrease in the amount of sebum was observed in the food supplement (Ca) group after 84 days of consumption, from 196.0±12.1 μg/cm² at DO to 185.1±9.9 μg/cm² at D56 and 171.7±11.4 μg/cm² at D84 (p=0.013), whereas there was no significant change in the food supplement (C′a) group, with 166.4±7.5 μg/cm² at DO to 168.0±7.0 μg/cm² at D56 and 168.5±7.4 μg/cm² at D84.

In addition, there was a significant difference between the food supplement (Ca) group and the food supplement (C′a) group at D84 (−11.9% vs+2.8%; p=0.044).

In addition, the processing of responses on the self-assessment form completed by the subjects of the study showed that 88% of subjects in the food supplement (Ca) group reported that their “hair was less greasy”, compared to 63% of subjects in the food supplement (C′a) group.

Through the clinical study, the composition (C) demonstrated its activity in limiting sebum production on the scalp.

### Illustrative Formulation Examples

TABLE 3
Clinical study capsule formula

| | Amount per |
|---|---|
| Component | capsule |
| Composition (C) | 15 mg |
| Maltodextrin | 230 mg |
| Dicalcium phosphate | 100 mg |
| Magnesium carbonate | 50 mg |
| Silica | 30 mg |
| Magnesium stearate | 10 mg |

TABLE 4
Capsule formula

| Component | Amount per capsule |
|---|---|
| Composition (C) | 30 mg |
| Maltodextrin | 220 mg |
| Beet juice | 70 mg |
| Magnesium | 15 mg |
| stearate | |

TABLE 5
Soft capsule formula

| Component | Amount per capsule | |
|---|---|---|
| Composition (C) | 70 | mg |
| Borage oil | 400 | mg |
| Glyceryl monostearate | 24.1 | mg |
| Vitamin C | 14.6 | mg |
| Lecithin | 14 | mg |
| Vitamin B3 | 2.9 | mg |
| Vitamin E | 2.7 | mg |
| Selenium | 2 | mg |
| Copper | 0.4 | mg |
| Vitamin B2 | 0.2 | mg |
| Biotin | 0.007 | mg |

### BIBLIOGRAPHY

(1) Lin et al. Morphogenesis, Growth Cycle and Molecular Regulation. Frontiers in cell and developmental Biology 2022, 10:899095. doi: 10.3389/fcell.2022.899095.
(2) Patent application FR3069160A1: Cosmetic or pharmaceutical composition for promoting hair growth, comprising a lyophilisate of dedifferentiated sea fennel cells.
(3) Nikfar & Jaberidoost. Hair in Encyclopedia of Toxicology (Third Edition), Academic Press, 2014, 813-815. https://doi.org/10.1016/B978-0-12-386454-3.00610-2.
(4) Patent application EP2821052A1: Combination of miliacin and polar lipids, particularly sphingolipids and/or phospholipids, for hair and scalp care.
(5) https://www.liperol.fr/le-cuir-chevelu/
(6) Makrantonaki et al. An update on the role of the sebaceous gland in the pathogenesis of acne. Dermato-Endocrinology 2011, 3:1, 41-49, doi: 10.4161/derm.3.1.13900.

1. A composition (C) comprising:
a wheat gluten extract comprising per 100% by mass:
from 20% by mass to 80% by mass of at least one sphingolipid (a1) resulting from amidation of a fatty acid with sphingosine,
from 10% by mass to 50% by mass of digalactosyldiacylglycerol (a2), and
from 1% by mass to 30% by mass of at least one phospholipid (a3), and wherein a sum of mass contents of the at least one sphingolipid (a1) resulting from the amidation of the fatty acid with sphingosine, the digalactosyldiacylglycerol (a2), and the at least one phospholipid (a3) is equal to 100% by mass, and
wherein the composition is in a form suitable for oral administration and is capable of limiting production of sebum on a scalp of a subject.

2. The composition (C) as claimed in claim 1, comprising per 100% by mass:
from 20% by mass to 60% by mass of the at least one sphingolipid (a1) resulting from the amidation of the fatty acid with sphingosine,
from 19% by mass to 50% by mass of the digalactosyldiacylglycerol (a2), and
from 1% by mass to 10% by mass of the at least one phospholipid (a3).

3. An edible pharmaceutical composition (Cp) comprising per 100% by mass:
from 10% by mass to 30% by mass of the composition (C) as claimed in claim 1, and
from 70% by mass to 90% by mass of at least one pharmaceutically acceptable edible technological additive,
wherein the edible pharmaceutical composition (Cp) is in a form suitable for oral administration and is capable of limiting production of sebum on a scalp of a subject.

4. The edible pharmaceutical composition (Cp) as claimed in claim 3, further comprising at least one active principle selected from the group consisting of bioactive lipids, water-soluble or water-dispersible trace element salts, water-soluble or fat-soluble vitamins, prebiotics, probiotics, milk proteins and/or milk protein concentrates, plant or animal enzymes, amino acids, peptides, sugars, flavor enhancers, and flavoring agents.

5. (canceled)

6. A method of cosmetic treatment for preventing or slowing an appearance of unsightly signs linked to a presence of excess sebum on a scalp of a subject in need thereof, the method comprising:
administering orally an effective amount to the subject of a composition (C) comprising, per 100% by mass:
from 20% by mass of at least one sphingolipid (a1) resulting from amidation of a fatty acid with sphingosine,
from 10% by mass of digalactosyldiacylglycerol (a2), and
from 1% by mass to 30% by mass of at least one phospholipid (a3),
wherein:
a sum of mass contents of the at least one sphingolipid (a1) resulting from the amidation of the fatty acid with sphingosine, the digalactosyldiacylglycerol (a2), and the at least one phospholipid (a3) being equal to 100% by mass, and
the composition (C) is a wheat gluten extract.

7. A food supplement (Ca) comprising per 100% by mass:
more than 0% by mass and less than 10% by mass of a composition (C) comprising per 100% by mass:
from 20% by mass to 80% by mass of at least one sphingolipid (a1) resulting from amidation of a fatty acid with sphingosine,
from 10% by mass to 50% by mass of digalactosyldiacylglycerol (a2), and
from 1% by mass to 30% by mass of at least one phospholipid (a3),
wherein:
a sum of mass contents of the at least one sphingolipid (a1) resulting from the amidation of the fatty acid with sphingosine, the digalactosyldiacylglycerol (a2), and the at least one phospholipid (a3) being equal to 100% by mass, and
the composition (C) is a wheat gluten extract, and
more than 90% by mass and less than 100% by mass of at least one nutritionally acceptable technological additive.

8. The food supplement (Ca) as claimed in claim 7, further comprising at least one active principle selected from the group consisting of bioactive lipids, water-soluble or water-dispersible trace element salts, water-soluble or fat-soluble vitamins, prebiotics, probiotics, milk proteins and/or milk protein concentrates, plant or animal enzymes, amino acids, peptides, sugars, flavor enhancers, and flavoring agents.

9. The food supplement (Ca) as claimed in claim 7, wherein the food supplement (Ca) is in a form of a tablet, a hard capsule, a soft capsule, a powder, a film-coated tablet, a confectionery gum, or a granule.

10. A method for preventing and/or slowing an appearance of unsightly signs linked to a presence of excess sebum on a scalp of a subject, the method comprising orally administering the food supplement (Ca) as claimed in claim 7 to the subject.

11. A method of cosmetic treatment for preventing or slowing an appearance of unsightly signs linked to a presence of excess sebum on a scalp of a subject, the method comprising orally administering an effective amount of the food supplement (Ca) as claimed in claim 7.

12. A method for slowing or limiting unsightly signs of an appearance of excess sebum on a scalp of a subject in need thereof, the method comprising administering an effective amount of the composition (C) as claimed in claim 1 to the subject.

13. A method for limiting the production of sebum on a scalp of a subject in need thereof, the method comprising administering an effective amount of the composition (C) as claimed in claim 1 to the subject.

14. The cosmetic treatment method as claimed in claim 6, wherein the composition (C) comprises, per 100% by weight, one or more of the following:
from 20% by weight to 60% by weight of the at least one sphingolipid (a1),
from 19% by weight to 50% by weight of the digalactosyldiacylglycerol (a2), and
from 1% by weight to 10% by weight of the at least one phospholipid (a3).

15. The food supplement (Ca) as claimed in claim 7, wherein, the composition (C) comprises, per 100% by weight, one or more of the following:
from 20% by weight to 60% by weight of the at least one sphingolipid (a1),
from 19% by weight to 50% by weight of the digalactosyldiacylglycerol (a2), and
from 1% by weight to 10% by weight of the at least one phospholipid (a3).
